(19) United States
(12) Patent Application Publication
(10) Pub. No.: US 2025/0025614 A1
(43) Pub. Date: Jan. 23, 2025

(54) MODULAR FLAT SHEET MEMBRANE FILTER

(71) Applicants: BAXTER INTERNATIONAL INC., Deerfield, IL (US); BAXTER HEALTHCARE SA, Glattpark (Opfikon) (CH)

(72) Inventors: Philipp KÜHN, Deerfield, IL (US); Ralf FLIEG, Deerfield, IL (US); Rainer BLICKLE, Deerfield, IL (US); Steffen WAGNER, Deerfield, IL (US)

(21) Appl. No.: 18/711,451

(22) PCT Filed: Nov. 21, 2022
(86) PCT No.: PCT/US2022/080247

§ 371 Date: May 17, 2024

Related U.S. Application Data
(60) Provisional application No. 63/291,073, filed on Dec. 17, 2021.

(51) Int. Cl. A61M 1/16

(52) U.S. Cl. CPC A61M 1/1672; A61M 2205/7518

(57) ABSTRACT

A peritoneal dialysis (“PD”) system (10) including a PD machine (20); a patient line (50) extending from the PD machine (20); and a filter set (100) in fluid communication with the patient line (50), the filter set (100) including a filter body (110, 150, 190) housing first and second filter membranes (112a, 112b, such as a sterilizing grade or bacteria reduction filter membranes), the filter body (110, 150, 190) configured to be placed in different arrangements such that fresh PD fluid (i) flows through the first filter membrane (112a) and then through the second filter membrane (112b) or (ii) splits and flows through first and second filter membranes (112a, 112b) in parallel.

### PRIORITY CLAIM

The present application claims priority to and the benefit of U.S. Provisional Application No. 63/291,073, filed on Dec. 17, 2021, the entire contents of which are hereby incorporated by reference.

### BACKGROUND

The present disclosure relates generally to medical fluid treatments and in particular to the filtering of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

PD fluid needs to be sterile or very near sterile because it is injected into the patient's peritoneal cavity, and is accordingly considered a drug. While bagged PD fluid is typically properly sterilized for treatment, PD fluid made online, or PD machines or cyclers that employ disinfection, may need additional sterilization.

There is accordingly a need for an effective, low cost way of providing additional sterilization to fresh PD fluid before it is delivered to a patient.

### SUMMARY

The present disclosure provides a peritoneal dialysis (“PD”) system having a PD machine or cycler that pumps fresh PD fluid through a patient line to a patient and removes used PD fluid from the patient via the patient line. The patient line may be reusable or disposable and in either case operates with and fluidly communicates with a filter set. If the patient line is reusable, the reusable patient line is connected to the filter set at the time of treatment. If the patient line is disposable, the filter set is merged into the disposable patient line in one embodiment. In either configuration a distal end of the filter set may be connected to the patient's transfer set, which in turn communicates fluidly with the patient's indwelling catheter.

The PD machine or cycler may include a durable PD fluid pump that pumps PD fluid through the pump itself without using a disposable component, or a disposable type PD fluid pump including a pump actuator that actuates a disposable, fluid-contacting pumping component, such as a peristaltic pump tube or a flexible pumping chamber. The PD machine or cycler also includes a plurality of valves, which may likewise be flow-through and durable without operating with a disposable component, or be disposable type valves having valve actuators that actuate a disposable, fluid-contacting valve component, such as a tube segment or a cassette-based valve seat.

The pumps and valves are under the automatic control of a control unit provided by the machine or cycler. In an embodiment, the valves include a fresh PD fluid valve that the control unit opens to allow the PD fluid pump to pump fresh PD fluid through a fresh PD fluid lumen of a dual lumen patient line to the patient. The valves also include a used PD fluid valve that the control unit opens to allow the PD fluid pump to pump used PD fluid from the patient through a used PD fluid lumen of the dual lumen patient line. It should be appreciated that while a single PD fluid pump may be used, dedicated fresh and used PD fluid pumps may be used alternatively. Also, a single PD fluid pump may include multiple pumping chambers for more continuous PD fluid flow.

The present disclosure sets forth multiple embodiments for a modular filter set. The modular filter sets may be configured to provide a redundant filter, a double membrane filter and a single membrane filter. In one primary embodiment, modular filter set includes filter membrane modules that are configured the same regardless of whether a redundant filter, a double membrane filter or a single membrane filter is provided. The membrane modules operate with different fluidic spacers that determine how fresh PD fluid is flowed. The fluidic spacers may for example be located, e.g., ultrasonically sealed, heat sealed and/or adhesively sealed, e.g., via solvent bonding, between two common membrane modules.

A redundant spacer causes fresh PD fluid entering the spacer to flow to one of the membrane modules, to be filtered through the filter membrane of that membrane module (first filtration), to flow to the second membrane module, to be filtered through the filter membrane of the second membrane module (redundant filtration), to flow out of the second membrane module back into the redundant spacer, and then to flow via a patient lumen of the redundant spacer to the patient. The overall redundant modular filter set is advantageous in that it provides the necessary sterilization even if one of the filter membranes has been compromised and provides double filtration of both filter membranes are intact.

A double membrane spacer instead causes fresh PD fluid entering the spacer to flow in two directions to both membrane modules simultaneously or in parallel, to be filtered in a split manner through both filter membranes simultaneously or in parallel, to flow out of both the first and second membrane modules back into the double membrane spacer, and then to flow via a patient lumen of the redundant spacer to the patient. The overall double membrane modular filter set is advantageous in that it effectively doubles the flow capacity of the filter set.

A single membrane spacer is used with a single membrane module. Here, fresh PD fluid entering the spacer flows into the membrane module, is filtered through the filter membrane of the membrane module, flows out of the membrane module back into the single membrane spacer, and then flows via a patient lumen of the single membrane spacer to the patient. The overall single membrane modular filter set is advantageous in that it reduces size and cost.

In a second primary embodiment, the modular filter set eliminates the spacers. Here again, the membrane modules are configured the same (at least initially) regardless of whether a redundant filter, a double membrane filter or a single membrane filter is provided. The membrane modules are here formed with openings (ports) and lumens or passageways needed for each of the redundant filter, double membrane filter and single membrane filter versions. A last manufacturing step is to plug the unused openings (ports) and not-needed lumens or passageways, so that a desired overall flowpath through the modular filter set is created.

A redundant modular filter set of the second primary embodiment is plugged so that fresh PD fluid entering one of the membrane modules is filtered through the filter membrane of that membrane module (first filtration), flows to the second membrane module, is filtered through the filter membrane of the second membrane module (redundant filtration), and flows out of the second membrane module via a patient lumen to the patient. The overall redundant modular filter set of the second primary embodiment is likewise advantageous because necessary sterilization is provided even if one of the filter membranes has been compromised and double filtration is provided if both filter membranes are intact.

A double membrane modular filter set of the second primary embodiment is instead plugged so that fresh PD fluid entering the one of the membrane modules flows first to a like compartment of the second membrane module, is filtered through both filter membranes simultaneously or in parallel, flows to a patient lumen in one of the membrane modules, and then flows via the patient lumen to the patient. The overall double membrane modular filter set of the second primary embodiment is likewise advantageous by effectively doubling the flow capacity of the filter set.

A single membrane modular filter set of the second primary embodiment uses a single membrane module. Here, the single membrane module is plugged so that fresh PD fluid entering the membrane module flows into an inlet compartment, is filtered through the filter membrane of the membrane module into an outlet compartment, and flows out of the outlet compartment via a patient lumen in the outlet compartment to the patient. The overall single membrane modular filter set of the second primary embodiment is likewise advantageous in that it reduces size and cost.

In a third primary embodiment, the modular filter set also eliminates the spacers and stacks the membrane modules. Inlet and outlet covers are also provided. The inlet cover includes a fresh PD fluid inlet. The outlet cover includes a patient lumen that carries filtered, fresh PD fluid to the patient and removes used PD fluid from the patient. After a desired number of membrane modules are stacked together (could be only a single module or two or more modules), the inlet and outlet covers are fitted to exposed surfaces of the one or more membrane module, and the modules and covers are ultrasonically sealed, heat sealed and/or adhesively sealed, e.g., via solvent bonding, to form the modular filter set of the third primary embodiment.

A redundant modular filter set of the third primary embodiment is formed so that fresh PD fluid entering one of the membrane modules of the stack via the inlet cover is filtered through the filter membrane of that membrane module (first filtration), flows to the second membrane module of the stack, is filtered through the filter membrane of the second membrane module (redundant filtration), and flows out of the second membrane module via the patient lumen of the outlet cover to the patient. The overall redundant modular filter set of the third primary embodiment is similarly advantageous because it mitigates a compromised filter membrane and provides double filtration if both filter membranes are intact.

A double membrane modular filter set of the third primary embodiment is instead formed so that fresh PD fluid entering through the inlet cover flows in parallel to both of the membrane modules, is filtered through both filter membranes simultaneously or in parallel, flows to a patient lumen of the outlet cover, and then flows via the patient lumen to the patient. The overall double membrane modular filter set of the third primary embodiment also advantageously doubles the flow capacity of the filter set.

A single membrane modular filter set of the third primary embodiment uses a single membrane module in combination with the inlet and outlet covers. Here, the inlet and outlet covers are sealed to the single membrane module so that fresh PD fluid entering the membrane module via the inlet cover flows into an inlet compartment of the membrane module, is filtered through the filter membrane of the membrane module into an outlet compartment of the membrane module, and flows out of the outlet compartment via a patient lumen of the outlet cover to the patient. The overall single membrane modular filter set of the second primary embodiment likewise reduces size and cost.

The membrane modules of the third primary embodiment may include different frames that are selected and stacked together to form redundant and double membrane modular filter sets. One of the frames forces all incoming fresh PD fluid to be filtered through the filter membrane fixed to the frame (used for redundant and single membrane modular filter sets). The other frame allows incoming fresh PD fluid to be split between (i) being filtered through the filter membrane fixed to the frame and (ii) bypassing the filter membrane and flowing through an opening to another membrane module (used for double membrane modular filter sets).

As mentioned above, the fresh and used PD fluid lumens of the dual lumen patient line may be reusable or disposable. In the instance in which the fresh and used PD fluid lumens are reusable, the lumens terminate with a patient line connector that connects to a lumen-side connector of each of the module filter sets discussed herein. The lumen-side connector in one embodiment includes a fresh PD fluid port for communication with the fresh PD fluid lumen of the dual lumen patient line and a used PD fluid port for communicating with the used PD fluid lumen of the dual lumen patient line. The lumen-side connector may also include threads for threadingly engaging mating threads of patient line connector. The threading of the patient line connector to the lumen-side connector seals mating ports of the patient line connector to the fresh and used PD fluid ports of the lumen-side connector in one embodiment, e.g., via one or more gasket.

Each of the modular filter sets discussed herein may also include a transfer set-side connector that connects to a short, flexible tube placed between the body of the filter set and the patient's transfer set, so that the generally rigid module filter set is spaced away from the generally rigid transfer set to aid patient comfort. That is, a directly abutted body of the filter set and transfer set may produce a combined rigid structure that is uncomfortable for the patient during sleep. The transfer set-side connector may simply be a tube port for sealingly accepting the short, flexible tube or be a threaded connector that threads to a mating connector provided at the end of the short, flexible tube.

For any of the modular filter sets discussed herein, used PD fluid removed through the patient's transfer set flows through the short, flexible tube, and the transfer set-side connector, patient lumen and the used PD fluid port of the lumen-side connector of the modular filter set under negative pressure via the PD fluid pump. Used PD fluid is pulled from the modular filter set, through the used PD fluid lumen of the dual lumen patient line, back to the PD machine or cycler. The PD machine or cycler pumps the used PD fluid under positive pressure to drain. The used PD fluid in each of the modular filter sets discussed herein generally does not contact the underside any the filter membrane. Effluent contact is at most minimal, such that the filter membranes of the present disclosure remain viable over the course of multiple fills of a treatment prior to being discarded with the filter set.

The hydrophilic nature of the filter membranes used for any of the modular filter sets discussed herein prevents air from migrating across the membrane once the membrane is fully wetted with fresh PD fluid and thus serves a secondary final stage air removal purpose. If needed however, it is contemplated to provide one or more hydrophobic membrane upstream of the upstream-most filter membrane (from a fresh PD fluid standpoint), e.g., adjacent to a fresh PD fluid inlet of the filter set. The one or more hydrophobic membrane allows air to be vented to atmosphere prior to the fresh PD fluid flowing through the filter membrane.

In light of the disclosure set forth herein, and without limiting the disclosure in any way, in a first aspect of the present disclosure, which may be combined with any other aspect, or portion thereof, a peritoneal dialysis (“PD”) system includes a PD machine; a patient line extending from the PD machine; and a filter set in fluid communication with the patient line, the filter set including a filter body housing first and second filter membranes, the filter body configured to be placed in different arrangements such that fresh PD fluid (i) flows through the first filter membrane and then through the second filter membrane or (ii) splits and flows through first and second filter membranes in parallel.

In a second aspect of the present disclosure, which may be combined with any other aspect, or portion thereof, the filter body includes a first membrane module housing the first filter membrane, a second membrane module housing the second filter membrane and a fluidic spacer in fluid communication with the first and second membrane modules, the fluidic spacer configured to cause (i) or (ii).

In a third aspect of the present disclosure, which may be combined with any other aspect, or portion thereof, the first membrane module is formed the same as the second membrane module.

In a fourth aspect of the present disclosure, which may be combined with any other aspect, or portion thereof, wherein for (i) the fluidic spacer is configured such that fresh PD fluid flows from the fluidic spacer through the first filter membrane of the first membrane module, flows into the second membrane module, flows through the second filter membrane, and flows back into the fluidic spacer before flowing to a patient.

In a fifth aspect of the present disclosure, which may be combined with any other aspect, or portion thereof, wherein for (ii) the fluidic spacer is configured such that fresh PD fluid splits from the fluidic spacer into the first and second membrane modules, is filtered through the first and second filter membranes in parallel, and flows back into the fluidic spacer before flowing to a patient.

In a sixth aspect of the present disclosure, which may be combined with any other aspect, or portion thereof, the fluidic spacer includes a patient lumen that allows filtered fresh PD fluid to flow to a patient and used PD fluid to flow from the patient.

In a seventh aspect of the present disclosure, which may be combined with any other aspect, or portion thereof, the fluidic spacer includes a blind fresh PD fluid inlet that accepts fresh PD fluid from the patient line.

In an eighth aspect of the present disclosure, which may be combined with any other aspect, or portion thereof, the filter body includes a first membrane module housing the first filter membrane, a second membrane module housing the second filter membrane, the first and second membrane modules including a plurality of inlets, passageways or lumens positioned and arranged to cause (i) and (ii), and wherein a portion of the inlets, passageways or lumens are plugged to cause (i) or (ii).

In a ninth aspect of the present disclosure, which may be combined with any other aspect, or portion thereof, the first membrane module is formed the same as the second membrane module.

In a tenth aspect of the present disclosure, which may be combined with any other aspect, or portion thereof, at least one of the first or second membrane modules includes an upper compartment and a lower compartment, the upper compartment formed the same as the lower compartment.

In an eleventh aspect of the present disclosure, which may be combined with any other aspect, or portion thereof, wherein for (i) the plurality of inlets, passageways or lumens are plugged such that fresh PD fluid flows through the first filter membrane of the first membrane module, flows into the second membrane module, and flows through the second filter membrane, before flowing to a patient.

In a twelfth aspect of the present disclosure, which may be combined with any other aspect, or portion thereof, for (ii) the plurality of inlets, passageways or lumens are plugged such that fresh PD fluid splits into the first and second membrane modules, and is filtered through the first and second filter membranes in parallel before flowing to a patient.

In a thirteenth aspect of the present disclosure, which may be combined with any other aspect, or portion thereof, the first and second membrane modules each include a patient lumen, and which for (ii) includes a bridge connector configured to divert fresh PD fluid from one of the patient lumens to the other of the patient lumens.

In a fourteenth aspect of the present disclosure, which may be combined with any other aspect, or portion thereof, the filter body includes a first membrane module housing the first filter membrane, a second membrane module housing the second filter membrane, the first and second membrane modules stacked together, the filter body further including an inlet cover and an outlet cover, at least one of the first and second membrane modules, the inlet cover and the outlet cover configurable to cause (i) or (ii).

In a fifteenth aspect of the present disclosure, which may be combined with any other aspect, or portion thereof, the first membrane module and the second membrane module employ at least one of a first frame that forces all incoming fresh PD fluid to be filtered through an associated first or second filter membrane or a second frame that filters a portion of the incoming fresh PD fluid through the associated first or second filter membrane and allows another portion of the incoming fresh PD fluid to bypass the associated first or second filter membrane.

In a sixteenth aspect of the present disclosure, which may be combined with any other aspect, or portion thereof, the patient line is a dual lumen patient line including a fresh PD fluid lumen placed in fluid communication with a fresh PD fluid port of the filter set, the dual lumen patient line further including a used PD fluid lumen placed in fluid communication with a used PD fluid port of the filter set.

In a seventeenth aspect of the present disclosure, which may be combined with any other aspect, or portion thereof, the filter set is configured to connect directly to a patient's transfer set, or wherein the filter set includes a flexible tube configured to connect to the patient's transfer set.

In an eighteenth aspect of the present disclosure, which may be combined with any other aspect, or portion thereof, the PD machine includes a pressure sensor positioned and arranged to sense the pressure of fresh PD fluid downstream from the filter membrane during a patient fill.

In a nineteenth aspect of the present disclosure, which may be combined with any other aspect, or portion thereof, the first and second filter membranes are sterilizing grade or bacteria reduction filter membranes.

In a twentieth aspect of the present disclosure, which may be combined with any other aspect, or portion thereof, a peritoneal dialysis (“PD”) system includes a PD machine; a patient line extending from the PD machine; and a filter set in fluid communication with the patient line, the filter set including a filter body and at least one of a first membrane or a second filter membrane, the filter body configured to be placed in different arrangements such that fresh PD fluid (i) flows through the first filter membrane and then through the second filter membrane, (ii) splits and flows through first and second filter membranes in parallel, or (iii) flows through the first filter membrane only.

In a twenty-first aspect of the present disclosure, which may be combined with any other aspect, or portion thereof, any of the features, functionality and alternatives described in connection with any one or more of FIGS. 1 to 17 may be combined with any of the features, functionality and alternatives described in connection with any other of FIGS. 1 to 17.

In light of the above aspects and the present disclosure discussed herein, it is an advantage of the present disclosure to provide a filter set that that is modular.

It is another advantage of the present disclosure to provide a filter set that is adaptable to meet different sterilization needs.

It is yet another advantage of the present disclosure to provide a filter set that may be configured to provide redundant disinfection.

It is a further advantage of the present disclosure to provide a filter set that filters fresh PD fluid and allows used PD fluid to pass without clogging the filter membrane.

It is still a further advantage of the present disclosure to provide a filter set having a filtration capacity that is readily adjustable to suit different pathogen load needs.

It is still another advantage of the present disclosure to provide a filter set that operates with a dual lumen patient line.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for peritoneal dialysis system including a dual lumen patient line operating with a modular filter set of the present disclosure.

FIG. 2 is a perspective view of a first primary embodiment for a modular patient line filter set of the present disclosure.

FIG. 3 is a perspective view of a redundant version of the first primary embodiment for a modular patient line filter set of the present disclosure.

FIG. 4 is a perspective view of a double filter membrane version of the first primary embodiment for a modular patient line filter set of the present disclosure.

FIG. 5 is a perspective view of a single filter membrane version of the first primary embodiment for a modular patient line filter set of the present disclosure.

FIG. 6 is a perspective view of a second primary embodiment for a modular patient line filter set of the present disclosure.

FIG. 7 is a perspective view of a redundant version of the second primary embodiment for a modular patient line filter set of the present disclosure.

FIG. 8 is a perspective view of a double filter membrane version of the second primary embodiment for a modular patient line filter set of the present disclosure.

FIG. 9 is a perspective view illustrating an embodiment for a bridge connector used with the double filter membrane version of the second primary embodiment for a modular patient line filter set of the present disclosure.

FIG. 10 is a perspective view of a single filter membrane version of the second primary embodiment for a modular patient line filter set of the present disclosure.

FIG. 11 is an assembled perspective view of a third primary embodiment for a modular patient line filter set of the present disclosure.

FIG. 12 is an exploded perspective view of the third primary embodiment for a modular patient line filter set of the present disclosure.

FIG. 13 is a perspective view of a membrane module having a first frame configured to filter all incoming fresh peritoneal dialysis (“PD”) fluid.

FIG. 14 is a perspective view of a membrane module having a second frame configured to filter some incoming fresh PD fluid and allow other incoming fresh PD fluid to bypass a filter membrane associated with the second frame.

FIG. 15 is a sectioned perspective view of a redundant version of the third primary embodiment for a modular patient line filter set of the present disclosure.

FIG. 16 is a sectioned perspective view of a double filter membrane version of the third primary embodiment for a modular patient line filter set of the present disclosure.

FIG. 17 is a sectioned perspective view of a single filter membrane version of the third primary embodiment for a modular patient line filter set of the present disclosure.

### DETAILED DESCRIPTION

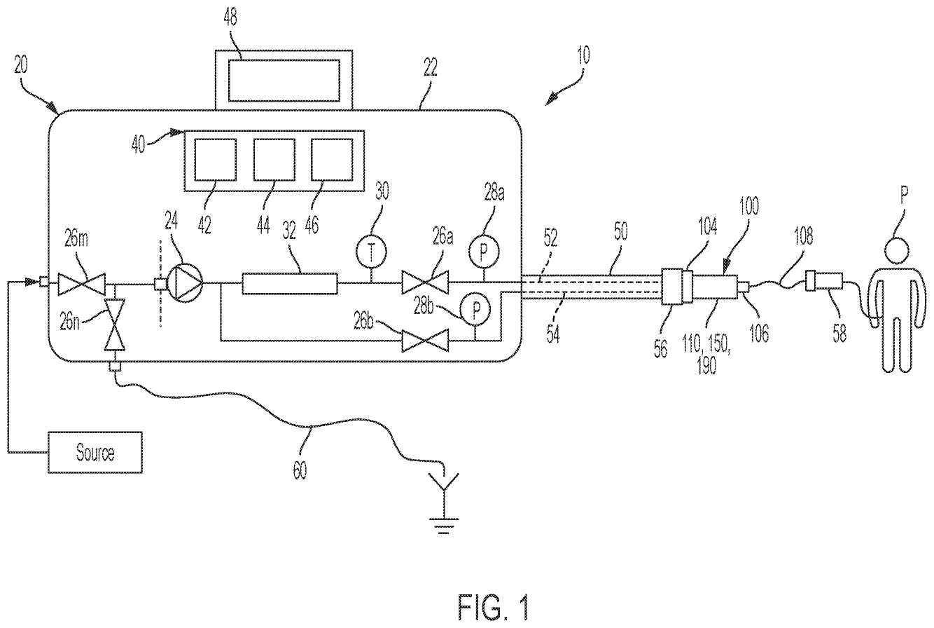

Referring now to the drawings and in particular to FIG. 1, a peritoneal dialysis (“PD”) system 10 is illustrated. PD system 10 includes a PD machine or cycler 20 that pumps fresh PD fluid through a patient line 50 to a patient P and removes used PD fluid from patient P via patient line 50. Patient line 50 may be reusable or disposable and in either case operates with and fluidly communicates with a modular filter set 100, which may alternatively be termed a bacterial reduction filter set. If patient line 50 is reusable, the reusable patient line is connected to modular filter set 100 at the time of treatment. If patient line 50 is instead disposable, modular filter set 100 is merged into or formed with disposable patient line 50 in one embodiment. In either configuration, a distal end of modular filter set 100 may be connected to the patient's transfer set 58, which in turn communicates fluidly with the indwelling catheter of patient P.

PD machine or cycler 20 may include a housing 22 providing a durable PD fluid pump 24 that pumps PD fluid through the pump itself without using a disposable component. Examples of durable pumps that may be used for PD fluid pump 24 include piston pumps, gear pumps and centrifugal pumps. Certain durable pumps, such as piston pumps are inherently accurate, so that machine or cycler 20 does not require additional volumetric control components. Other durable pumps, such as gear pumps and centrifugal pumps may not be as accurate, such that machine or cycler 20 provides a volumetric control device such as one or more flowmeter (not illustrated).

Pump 24 may alternatively be a disposable type PD fluid pump, which includes a pump actuator that actuates a disposable, fluid-contacting pumping component, such as a peristaltic pump tube or a flexible pumping chamber. Examples of disposable PD fluid pumps that may be used for PD fluid pump 24 include rotary or linear peristaltic pump actuators that actuate tubing, pneumatic pump actuators that actuate cassette sheeting, electromechanical pump actuators that actuate cassette sheeting and platen pump actuators that actuate tubing. It should be appreciated that while a single PD fluid pump 24 may be used, dedicated fresh and used PD fluid pumps may be used alternatively. Also, single PD fluid pump 24 may include multiple pumping chambers for more continuous PD fluid flow.

PD machine or cycler 20 also includes a plurality of valves 26a, 26b, 26m, 26n, which may likewise be flow-through and durable without operating with a disposable component, or be disposable type valves having valve actuators that actuate a disposable, fluid-contacting valve component, such as a tube segment or a cassette-based valve seat. Examples of durable valves that may be used for valves 26a, 26b, 26m, 26n include flow-through solenoid valves. Such valves may be two-way or three-way valves. Examples of disposable valves that may be used for valves 26a, 26b, 26m, 26n include solenoid pinch valves that pinch closed flexible tubing, pneumatic valve actuators that actuate cassette sheeting, and electromechanical valve actuators that actuate cassette sheeting.

Machine or cycler 20 likely includes many valves 26a to 26n. For ease of illustration, machine or cycler 20 is shown having a fresh PD fluid valve 26a that is controlled to open to allow PD fluid pump 24 to pump fresh PD fluid under positive pressure through a fresh PD fluid lumen 52 of dual lumen patient line 50 to patient P. The valves also include a used PD fluid valve 26b that is controlled to open to allow PD fluid pump 24 to pull used PD fluid from patient P under negative pressure through a used PD fluid lumen 54 of dual lumen patient line 50. Valve 26m is provided to allow selective access to one or more PD fluid source, while valve 26n is provided to allow selective access via a drain line 60 to a drain, such as a drain container or house drain.

Machine or cycler 20 in the illustrated embodiment also includes pressure sensors, such as pressure sensors 28a, 28b. Pressure sensor 28a is located just downstream from fresh PD fluid valve 26a, while pressure sensor 28b is located just upstream from used PD fluid valve 26b. Pressure sensor 28a may accordingly sense the pressure in fresh PD fluid lumen 52 of dual lumen patient line 50 even if fresh PD fluid valve 26a is closed, while pressure sensor 28b may sense the pressure in used PD fluid lumen 54 of dual lumen patient line 50 even if used PD fluid valve 26b is closed. Additionally, pressure sensor 28a is positioned to sense the pressure of fresh PD fluid upstream from the filter membrane discussed herein during a patient fill. Pressure sensor 28b perhaps more importantly is positioned to sense the pressure of fresh PD fluid downstream from the filter membrane discussed herein during a patient fill, which therefore takes into account the pressure drop due to one multiple (if redundant).

Pump 24 and valves 26a, 26b in the illustrated embodiment are under the automatic control of control unit 40 provided by machine or cycler 20 of system 10, while pressure sensors 28a, 28b (and other sensors) output to control unit 40. Control unit 40 in the illustrated embodiment includes one or more processor 42, one or more memory 44 and a video controller 46. Control unit 40 receives, stores and processes signals or outputs from pressure sensors 28a, 28b, and other sensors provided by machine or cycler 20, such as one or more temperature sensor 30 and one or more conductivity sensor (not illustrated). Control unit 40 may use pressure feedback from one or more of pressure sensor 28a, 28b to control PD fluid pump 24 to pump dialysis fluid at a desired pressure or within a safe pressure limit (e.g., within 0.21 bar (three psig) of positive pressure to a patient's peritoneal cavity and −0.10 bar (−1.5 psig) of negative pressure from the patient's peritoneal cavity).

Control unit 40 uses temperature feedback from one or more temperature sensor 30 for example to control a heater 32, such as an inline heater, to heat fresh PD fluid to a desired temperature, e.g., body temperature or 37° C. In one embodiment, heater 32 is used additionally to heat a disinfection fluid, such as fresh PD fluid, to disinfect PD fluid pump 24, valves 26a to 26n, heater 32 and all reusable fluid lines within machine or cycler 20 to ready the machine or cycler for a next treatment. The additional filtration discussed herein provides a layer of protection in addition to the heated fluid disinfection to ensure that the PD fluid is safe for delivery to patient P.

Video controller 46 of control unit 40 interfaces with a user interface 48 of machine or cycler 20, which may include a display screen operating with a touchscreen and/or one or more electromechanical button, such as a membrane switch. User interface 48 may also include one or more speaker for outputting alarms, alerts and/or voice guidance commands. User interface 48 may be provided with the machine or cycler 20 as illustrated in FIG. 1 and/or be a remote user interface operating with control unit 40. Control unit 40 may also include a transceiver (not illustrated) and a wired or wireless connection to a network, e.g., the internet, for sending treatment data to and receiving prescription instructions from a doctor's or clinician's server interfacing with a doctor's or clinician's computer.

Referring still to FIG. 1, modular filter set 100 in one embodiment includes a lumen-side connector 104, a transfer set-side connector 106, optionally a short, flexible tube 108 and a filter body 110, 150 or 190. Short, flexible tube 108 is located between filter body 110, 150 or 190 and the patient's transfer set 58, so that generally rigid filter body 110, 150 or 190 of filter set 100 is spaced away from generally rigid transfer set 58 to aid patient comfort. That is, a directly abutted filter body 110, 150 or 190 and transfer set 58 may produce a combined rigid structure that is uncomfortable for the patient during sleep.

Lumen-side connector 104, transfer set-side connector 106 and filter body 110, 150 or 190 may be molded together from one or more pieces ultrasonically, via heat seal and/or adhesively, e.g., via solvent bonding. Any of lumen-side connector 104, transfer set-side connector 106, optional short, flexible tube 108 and filter body 110, 150 or 190 may be made of any one or more plastic, such as, polystyrene (“PS”), polycarbonate (“PC”), blends of polycarbonate and acrylonitrile-butadiene-styrene (“PC/ABS”), polyvinyl chloride (“PVC”), polyethylene (“PE”), polypropylene (“PP”), polyesters like polyethylene terephthalate (“PET”), or polyurethane (“PU”).

Lumen-side connector 104 may include a fresh PD fluid port (not illustrated) for communicating with fresh PD fluid lumen 52 of dual lumen patient line 50 and a used PD fluid port (not illustrated) for communicating with used PD fluid lumen 54 of dual lumen patient line 50. The fresh and used PD fluid ports may be surrounded by a shroud (not illustrated) of lumen-side connector 104, wherein the shroud may be formed with threads (not illustrated) for threadingly engaging mating threads of patient line connector 56. The threading of patient line connector 56 to lumen-side connector 104 seals mating ports (not illustrated) of patient line connector 56 to the fresh and used PD fluid ports of lumen-side connector 104 in one embodiment, e.g., via one or more compressible gasket (not illustrated), such as a silicone or other suitable rubber gasket. The shroud of lumen-side connector 104 may be formed with a key. Patient line connector 56 is formed with a mating key so that the patient line connector can only be introduced into the shroud in a proper orientation, aligning fresh PD fluid lumen 52 with the fresh PD fluid port and used PD fluid lumen 54 with the used PD fluid port of lumen-side connector 104. If dual lumen patient line 50 is disposable, lumen-side connector 104 may alternatively simply include ports, e.g., fresh and used PD fluid ports to which fresh and used PD fluid lumens 52 and 54 respectively extend over for sealing to the ports in any manner described herein.

Transfer set-side connector 106 either connects directly to a mating connector of the patient's transfer set 58 (if no tube 108 is provide) or to a mating connector of a short, flexible tube 108 placed between filter housing 102 and the patient's transfer set 58. Transfer set-side connector 106 may include a port and threaded shroud (not illustrated) for a luer type connection to a mating connector. Transfer set-side connector 106 may alternatively simply be a port to which short, flexible tube 108 extends over for sealing to the port in any manner described herein.

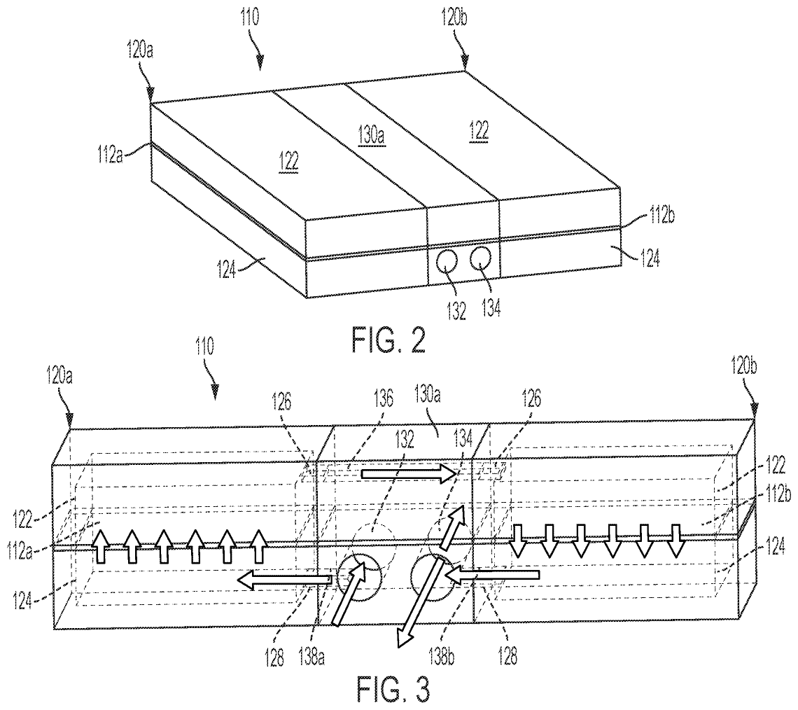

Referring now to FIGS. 2 to 5, filter body 110 in a first primary embodiment of the present disclosure is illustrated in more detail. Filter body 110 is shown in a generally rectangular format but may have any desired and/or optimized shape. Filter body 110 in the illustrated embodiment includes a pair of membrane modules 120a, 120b. Membrane modules 120a, 120b in one embodiment are constructed the same to aid in the modularity of filter set 100 and system 10.

FIG. 2 illustrates that membrane modules 120a, 120b each include an upper compartment 122 and a lower compartment 124. A filter membrane 112a is located between upper compartment 122 and lower compartment 124 of membrane module 120a, such that fresh PD fluid in upper compartment 122 is separated from fresh PD fluid in lower compartment 124 by filter membrane 112a. A filter membrane 112b is likewise located between upper compartment 122 and lower compartment 124 of membrane module 120b, such that fresh PD fluid in upper compartment 122 is separated from fresh PD fluid in lower compartment 124 by filter membrane 112b.

Filter membranes 112a and 112b in the illustrated embodiment are provided in the form of a flat sheet that may bisect membrane modules 120a, 120b respectively. Filter membranes 112a, 112b may be sterilizing grade or bacteria reduction hydrophilic membranes, which may be formed with porous walls having a pore size of about 0.2 micron through which the fresh PD fluid flows for further filtration. Filter membranes 112a, 112b may be made of, for example, polysulfone or polyethersulfone blended with polyvinylpyrrolidone. As illustrated below, filter membranes 112a, 112b are formed such that fresh PD fluid may be filtered through the membranes in either direction, top to bottom or bottom to top.

The hydrophilic nature of filter membranes 112a, 112b prevents air from migrating across the membrane once the membrane is fully wetted with fresh PD fluid and thus serves a secondary final stage air removal purpose. If needed however, it is contemplated for any embodiment described herein to provide one or more hydrophobic membrane (not illustrated) upstream of first filter membrane 112a (from a fresh PD fluid standpoint). The one or more hydrophobic membrane allows air to be vented to atmosphere prior to the fresh PD fluid flowing through any of filter membranes 112a, 112b. The hydrophobic membrane may be constructed for example from polytetrafluoroethylene (“PTFE”).

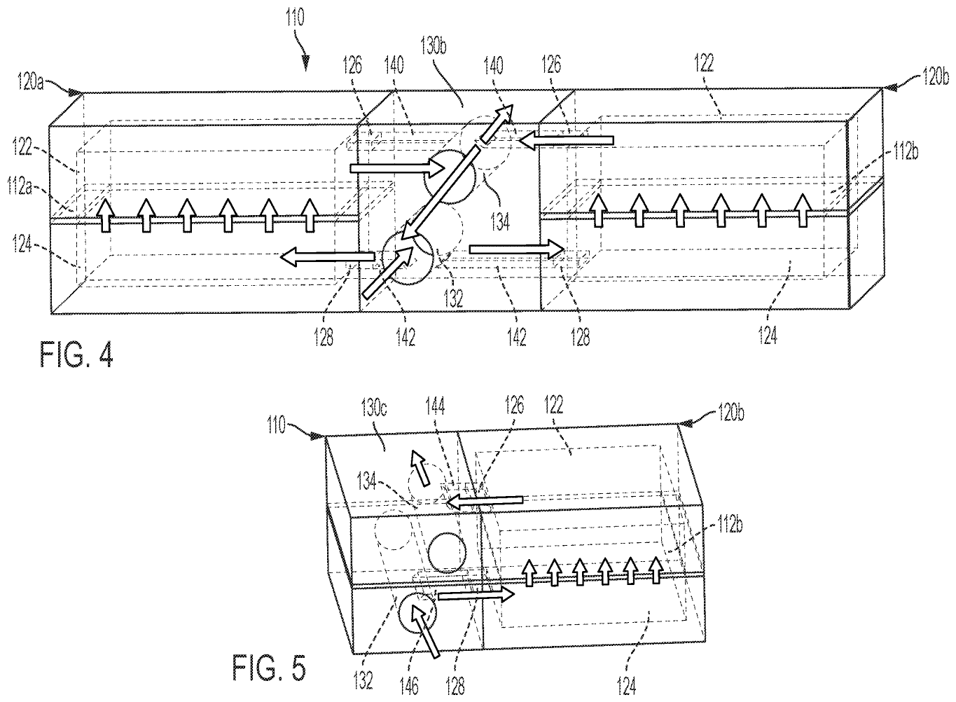

FIGS. 3 and 4 both illustrate that fluid passageways 126 extend from upper compartments 122 of membrane modules 120a, 120b through an outer wall of the upper compartments. Fluid passageways 128 extend from lower compartments 124 of membrane modules 120a, 120b through an outer wall of the lower compartments. Fresh PD fluid may therefore flow into or out of upper compartments 122 and lower compartments 124 via fluid passageways 126 and 128 respectively.

Different fresh PD flowpaths taken through modular filter body 110 are provided by differently configured fluidic spacers 130a to 130c. Fluidic spacer 130a is illustrated in FIGS. 2 and 3, while fluidic spacer 130b is illustrated in FIG. 4 and fluidic spacer 130c is illustrated in FIG. 5. When a desired fluidic spacer 130a to 130c is selected, upper and lower compartments 122 and 124 along with filter membranes 112a and 112b (FIG. 5 only includes filter membrane 112b) and the rest of modular filter set 100 may be sealed together at once, for example, ultrasonically sealed, heat sealed and/or adhesively sealed, e.g., via solvent bonding. Modular filter set 100 may alternatively be formed in steps, e.g., membrane modules 120a, 120b are formed separately and are then ultrasonically sealed, heat sealed and/or adhesively sealed, e.g., via solvent bonding, to a selected fluidic spacer 130a to 130c.

Fluidic spacer 130a as illustrated in FIG. 3 includes or defines a fresh PD fluid inlet 132 and a patient lumen 134. Fresh PD fluid inlet 132 may for example extend from or fluidly communicate with the fresh PD fluid port of lumen-side connector 104, while patient lumen 134 may for example extend from or fluidly communicate with the used PD fluid port of lumen-side connector 104. Fresh PD fluid inlet 132 in the illustrated embodiment is formed as a blind lumen that does not extend all the way through the back of fluidic spacer 130a. Patient lumen 134 on the other hand does extend all the way through the back of fluidic spacer 130a, e.g., to transfer set-side connector 106.

Fluidic spacer 130a as illustrated in FIG. 3 further includes or defines three fluid channels that set the direction of fresh PD fluid flow through modular filter body 110. Fluidic spacer 130a includes or defines an upper cross channel 136 that extends between and allows fresh PD fluid through between fluid passageways 126 formed in upper compartments 122 of membrane modules 120a, 120b. Fluidic spacer 130a includes or defines lumen channels 138a and 138b. Lumen channel 138a extends between and allows fresh PD fluid to flow between fluid passageway 128 of membrane module 120a and fresh PD fluid inlet 132. Lumen channel 138b extends between and allows fresh PD fluid to flow between fluid passageway 128 of membrane module 120b and patient lumen 134.

Fluidic spacer 130a is a redundant spacer that causes fresh PD fluid entering the spacer through fresh PD fluid inlet 132 to flow through lumen channel 138a and fluid passageway 128 into lower compartment 124 of membrane module 120a. Fresh PD fluid is then filtered through filter membrane 112a into upper compartment 122 of membrane module 120a (first filtration). Once filtered fresh PD fluid then flows through fluid passageways 126 and cross channel 136 into upper compartment 122 of membrane module 120b. Once filtered fresh PD fluid is then filtered again through filter membrane 112b into lower compartment 124 of membrane module 120b (redundant filtration). Twice filtered fresh PD fluid then flows out of lower compartment 124 of membrane module 120b through lumen channel 138b into patient lumen 134, and from patient lumen 134 to the patient for a patient fill of a PD treatment. The overall redundant modular filter set 100 is advantageous in that it provides the necessary sterilization even if one of the filter membranes 112a, 112b has been compromised and provides double filtration if both filter membranes 112a, 112b are intact.

Fluidic spacer 130b as illustrated in FIG. 4 also includes or defines fresh PD fluid inlet 132 and patient lumen 134. Fresh PD fluid inlet 132 is again formed as a blind lumen that does not extend all the way through the back of fluidic spacer 130b. Patient lumen 134 on the other hand does extend all the way through the back of fluidic spacer 130a, e.g., to transfer set-side connector 106. Fluidic spacer 130b as illustrated in FIG. 4 includes or defines two fluid channels that set the direction of fresh PD fluid flow through modular filter body 110. Fluidic spacer 130b includes or defines lumen channel 140 that extends between and allows fresh PD fluid to flow between patient lumen 134 and both fluid passageways 126 of both membrane modules 120a, 120b. Fluidic spacer 130b also includes or defines lumen channel 142 that extends between and allows fresh PD fluid to flow between PD fluid inlet 132 and both fluid passageways 128 of both membrane modules 120a, 120b.

Fluidic spacer 130b is a double membrane spacer that instead causes fresh PD fluid entering the spacer via PD fluid inlet 132 to be split and flow in two opposite directions to lower compartments 124 of both membrane modules 120a, 120b simultaneously or in parallel via lower lumen channels 142 and fluid passageways 128. Fresh PD fluid is then filtered from lower compartments 124 through both filter membranes 112a, 112b simultaneously or in parallel into upper compartments 122 of both membrane modules 120a, 120b. Filtered fresh PD fluid then flows through lumen channel 140 into patient lumen 134, and from patient lumen 134 to the patient for a patient fill of a PD treatment. Double membrane modular filter set 100 is advantageous in that it effectively doubles the flow capacity of the filter set compared to the same sized filter set using fluidic spacer 130a.

Fluidic spacer 130c as illustrated in FIG. 5 operates with only a single membrane module 120b. Fluidic spacer 130c also includes or defines fresh PD fluid inlet 132 and patient lumen 134. Fresh PD fluid inlet 132 is again formed as a blind lumen that does not extend all the way through the back of fluidic spacer 130b. Patient lumen 134 on the other hand does extend all the way through the back of fluidic spacer 130a, e.g., to transfer set-side connector 106. A lumen channel 144 extends between and allows fresh PD fluid communication between patient lumen 134 and upper compartment 122 of membrane module 120b. A second lumen channel 146 extends between and allows fresh PD fluid communication between PD fluid inlet 132 and lower compartment 124 of membrane module 120b.

Fluidic spacer 130c is a single membrane spacer operating with single membrane module 102b. Here, fresh PD fluid entering single membrane spacer 130c via PD fluid inlet 132 flows into lower compartment 124 of membrane module 120b via lumen channel 146 and fluid passageway 128. The fresh PD fluid is then filtered through filter membrane 112b into upper compartment 122. Filtered fresh PD fluid then flows via fluid passageway 126 and lumen channel 144 into patient lumen 134, and from patient lumen 134 to the patient for a patient fill of a PD treatment. Single membrane modular filter set 100 is advantageous in that it reduces size and cost.

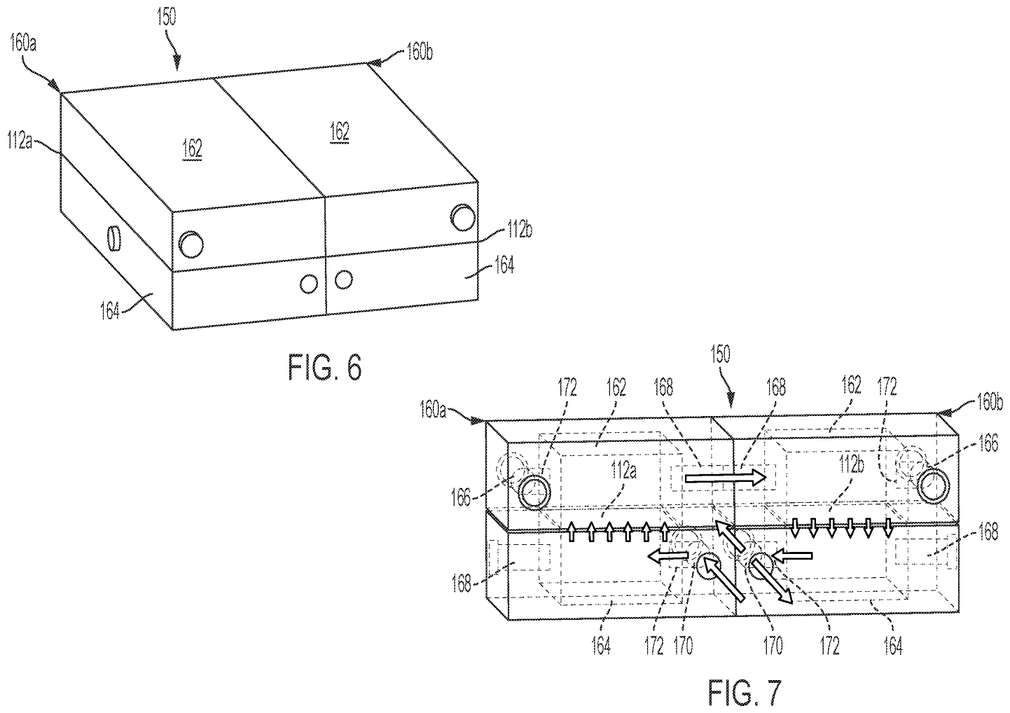

Referring now to FIGS. 6 to 10, filter body 150 of a second primary embodiment of the present disclosure is illustrated in more detail. Filter body 150 is shown in a generally rectangular format but may have any desired and/or optimized shape. Filter body 150 in the illustrated embodiment includes a pair of membrane modules 160a, 160b. Membrane modules 160a, 160b in one embodiment are constructed the same (at least initially), regardless of whether a redundant filter, a double membrane filter or a single membrane filter is provided, to aid in the modularity of filter set 100 and system 10. Filter body 150 eliminates spacers 130a to 130c associated with filter body 110.

Membrane modules 160a, 160b are formed with openings (ports) and lumens or passageways needed for each of the redundant filter, double membrane filter and single membrane filter versions. A last or later manufacturing step for modules 160a, 160b is to plug the unused openings (ports) and not-needed lumens or passageways, so that a desired overall flowpath through the modular filter set 100 is created.

FIG. 6 illustrates that membrane modules 160a, 160b each include an upper compartment 162 and a lower compartment 164. A filter membrane 112a is located between upper compartment 162 and lower compartment 164 of membrane module 160a, such that fresh PD fluid in upper compartment 162 is separated from fresh PD fluid in lower compartment 164 by filter membrane 112a. A filter membrane 112b is likewise located between upper compartment 162 and lower compartment 164 of membrane module 160b, such that fresh PD fluid in upper compartment 162 is separated from fresh PD fluid in lower compartment 164 by filter membrane 112b. Filter membranes 112a, 112b are again formed such that fresh PD fluid may be filtered through the membranes in either direction, top to bottom or bottom to top.

FIGS. 6 and 7 both illustrate the openings (ports) and lumens or passageways needed for each of the redundant filter, double membrane filter and single membrane filter versions. In particular, upper compartment 162 of membrane modules 160a, 160b in the illustrated embodiment includes a fresh PD fluid inlet 166. Fresh PD fluid inlets 162 may for example extend from or fluidly communicate with the fresh PD fluid port of lumen-side connector 104. Fresh PD fluid inlets 162 in the illustrated embodiment may be formed as (i) blind lumens that do not extend all the way through the back of the membrane modules (where modules 160a, 160b are formed as left and right pairs) or (ii) as through-hole lumens where a backside end of the lumen is plugged or blocked, effectively forming a blind lumen. Upper compartment 162 of membrane modules 160a, 160b in the illustrated embodiment also includes fluid passageways 168 that allow fresh PD fluid in upper compartments 162 to communicate fluidly with each other. Note that lower compartments 164 also include fluid passageways 168, which are always plugged in the lower compartment, but which may be unplugged when the module piece is used instead as an upper compartment 162, allowing a single module piece to be produced.

Lower compartment 164 of membrane modules 160a, 160b in the illustrated embodiment includes a patient lumen 170. Patient lumen 170 extends all the way through lower compartment 164 and may operate as a fresh PD fluid inlet from patient line 50 or as a fresh PD fluid outlet to the patient, e.g., communicating fluidly with transfer set-side connector 106. Patient lumen 170 may further be used to carry used PD fluid removed from the patient. Lower compartment 164 of membrane modules 160a, 160b in the illustrated embodiment also includes a fluid passageway 172 that extends from patient lumen 170 to the fluid holding area of lower compartment 164, allowing fresh PD fluid to flow in either direction between patient lumen 170 and the lower compartment. Fluid passageways 172 are also provided in upper compartment 162 extending from fresh PD fluid inlet 166 to the fluid holding area of upper compartment 162, allowing fresh PD fluid to flow in either direction between PD fluid inlet 166 and the upper compartment.

It should be appreciated that a single molded piece may operate as any of the four separate pieces of filter body 150 illustrated in FIGS. 6 and 7. Once filter membranes 112a and 112b are inserted between upper and lower compartments 162, 164, and the necessary plugs are inserted, e.g., adhered, the four separate pieces may be sealed together at once, for example, ultrasonically, heat sealed and/or adhesively sealed, e.g., via solvent bonding. Modular filter set 100 may alternatively be formed in steps, e.g., membrane modules 160a, 160b are formed separately with plugs in place and are then ultrasonically, heat sealed and/or adhesively sealed, e.g., via solvent bonding, together.

FIGS. 6 and 7 illustrate a redundant configuration for filter body 150. Here, fresh PD fluid inlets 166 in upper compartments 162 and fluid passageways 168 in lower compartment 164 for both modules 160a, 160b are fully plugged (fresh PD fluid inlets 166 plugged to at least cover fluid passageways 172). The back half of patient lumen 170 in membrane module 160a is plugged, but so as not to cover fluid passageway 172. Patient lumen 170 in membrane module 160b and fluid passageways 168 in upper compartments 162 are left unplugged and fully open.

Such configuration causes fresh PD fluid entering patient lumen 170 in membrane module 160a to flow through fluid passageway 172 into lower compartment 164 of membrane module 160a. Fresh PD fluid is then filtered through filter membrane 112a into upper compartment 162 of membrane module 160a (first filtration). Once filtered fresh PD fluid then flows through fluid passageways 168 of both upper compartments and into the fluid holding portion of 162 of membrane module 160b. Once filtered fresh PD fluid is then filtered again through filter membrane 112b into lower compartment 164 of membrane module 160b (redundant filtration). Twice filtered fresh PD fluid then flows out of lower compartment 164 of membrane module 160b through fluid passageway 172 into patient lumen 170 in membrane module 160b, and from patient lumen 170 to the patient for a patient fill of a PD treatment. It should be appreciated that while fresh PD fluid in patient lumen 170 in membrane module 160b is theoretically able to flow out the front of patient lumen 170 into used PD fluid lumen 54, used PD fluid lumen 54 is a closed, static line due to the closure of used PD fluid valve 26b, forcing the fresh PD fluid to flow in the opposite direction towards the patient. The overall redundant modular filter set 100 using filter body 150 is advantageous in that it provides the necessary sterilization even if one of the filter membranes 112a, 112b has been compromised and provides double filtration if both filter membranes 112a, 112b are intact.

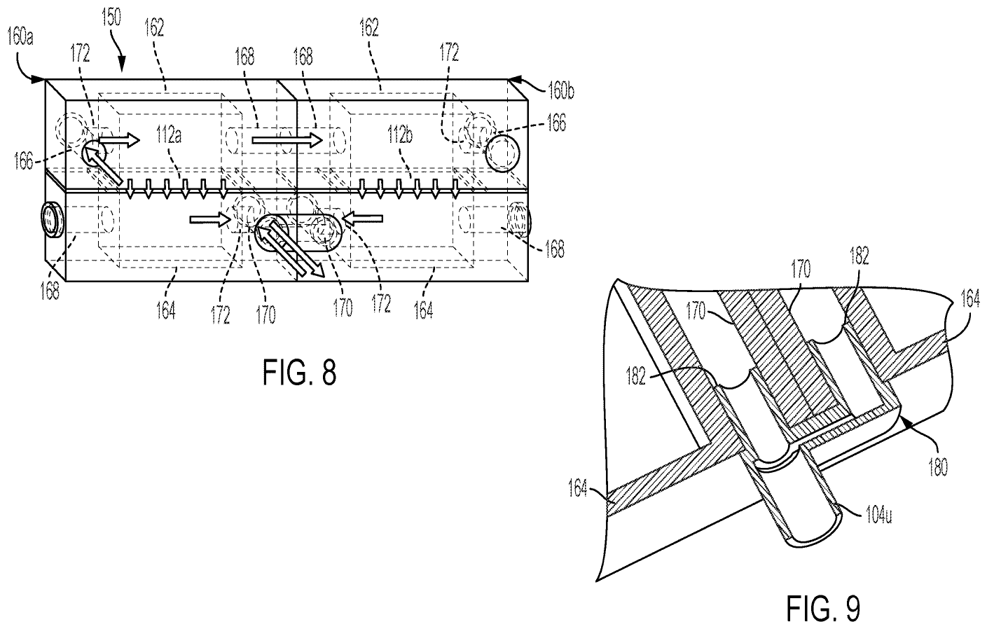

FIGS. 8 and 9 illustrate a double membrane configuration for filter body 150. Here, fresh PD fluid inlet 166 in upper compartment 162 of membrane module 160b and fluid passageways 168 in lower compartment 164 of both membrane modules 160a, 160b are fully plugged (fresh PD fluid inlet 166 of membrane module 160b is plugged to at least cover fluid passageway 172). The back half of patient lumen 170 in membrane module 160b is plugged, but so as not to cover fluid passageways 172. Fluid passageways 168 in upper compartments 162 of both membrane modules 160a, 160b are left unplugged and fully open.

The fronts of patient lumens 170 in membrane modules 160a and 160b are fitted with a bridge connector 180 illustrated in FIG. 9. Bridge connector 180 is formed, e.g., molded, from any of the materials described herein. Bridge connector 180 includes legs 182 that are ultrasonically sealed, heat sealed and/or adhesively sealed, e.g., via solvent bonding, to the inner surfaces of patient lumens 170. The common port of bridge connector 180 forms the used PD fluid port 104u of lumen-side connector 104 (FIG. 1). Bridge connector 180 allows fresh PD fluid to flow from patient lumen 170 of membrane module 160b into patient lumen 170 of membrane module 160a, so that all fresh PD flows out the back of patient lumen 170 of membrane module 160a to the patient. Here again, while it is theoretically possible for fresh PD fluid to flow out used PD fluid port 104u into used PD fluid lumen 54, used PD fluid lumen 54 is a closed, static line due to the closure of used PD fluid valve 26b, forcing the fresh PD fluid to flow in the opposite direction towards the patient.

The double membrane configuration of FIGS. 8 and 9 causes fresh PD fluid entering via fresh PD fluid inlet 166 of membrane module 160a to flow through upper compartment 162 of membrane module 160a and upper fluid passageways 168 into upper compartment 162 of membrane module 160b. Fresh PD fluid pressurizes within both upper compartment 162 and is then filtered through both filter membranes 112a, 112b simultaneously or in parallel into lower compartments 164 of both membrane modules 160a, 160b. Filtered fresh PD fluid then flows through fluid passageways 172 into patient lumens 170, and via bridge connector 180 as discussed above from patient lumens 170 to the patient for a patient fill of a PD treatment. Double membrane modular filter set 100 using filter body 150 is advantageous because it effectively doubles the flow capacity of the filter set.

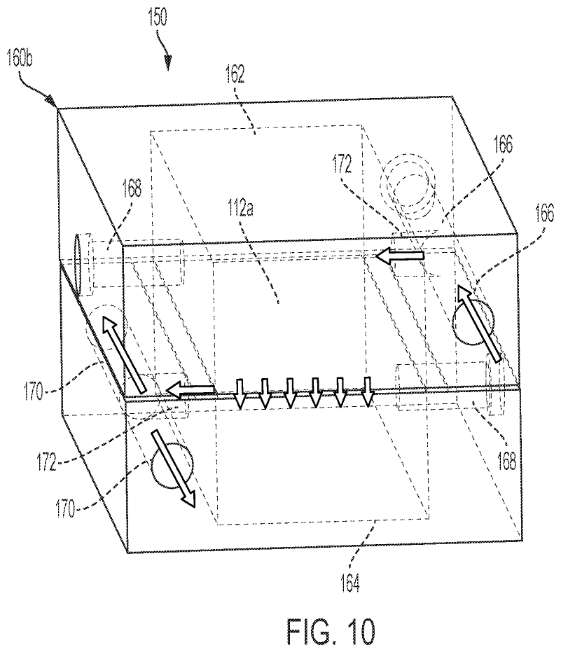

FIG. 10 illustrates a single membrane configuration for filter body 150. Here, only a single membrane module, e.g., membrane module 160b, is used. Fluid passageways 168 in upper compartment 162 and lower compartment 164 are plugged. A back or rear portion of fresh PD fluid inlet 166 in upper compartment 162 is also plugged but so as to leave fluid passageway 172 open. Patient lumen 170 in lower compartment 174 is fully open all the way through the lower compartment to allow used PD fluid to flow from the patient into used PD fluid lumen 54.

The single membrane configuration of FIG. 10 causes fresh PD fluid entering fresh PD fluid inlet 166 in upper compartment 162 to flow through fluid passageway 172 into the fluid receiving portion of upper compartment 162. The fresh PD fluid is then filtered through filter membrane 112b into lower compartment. Filtered fresh PD fluid then flows via fluid passageway 172 into patient lumen 170, and from patient lumen 170 to the patient for a patient fill of a PD treatment. Here again, while it is theoretically possible for fresh PD fluid to flow out patient lumen 170 into used PD fluid lumen 54, used PD fluid lumen 54 is a closed, static line due to the closure of used PD fluid valve 26b, forcing the fresh PD fluid to flow in the opposite direction towards the patient. Single membrane modular filter set 100 using filter body 150 is advantageous in that it reduces size and cost.

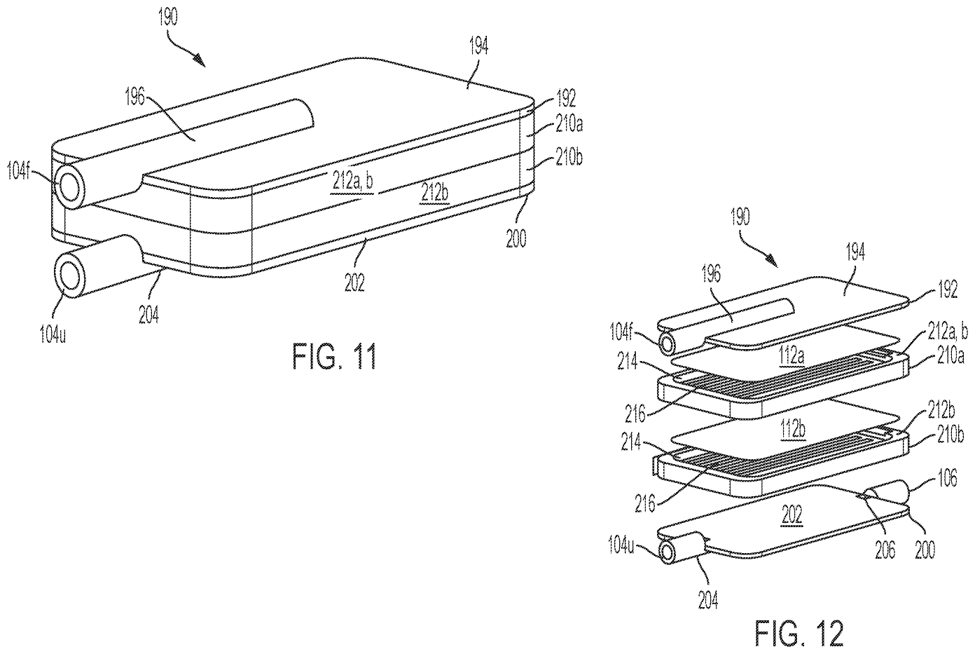

Referring now to FIGS. 11 to 17, filter body 190 of a third primary embodiment of the present disclosure is illustrated in more detail. FIGS. 11 and 12 illustrate that in the third primary embodiment using filter body 190, modular filter set 100 eliminates spacers 130a to 130c associated with filter body 110 and stacks membrane modules 210a, 210b . . . 210n. Inlet and outlet covers 192, 200 are also provided. Inlet cover 192 includes or defines a shell 194 providing a space for fresh PD fluid to be distributed across the shell before being filtered through a filter membrane. A fresh PD fluid inlet 196 is formed with shell 194 and extends to a fresh PD fluid port 104f of lumen-side connector 104 (FIG. 1). Although not viewable in FIG. 11 or 12, shell 194 defines an opening that allows fresh PD fluid to flow from fresh PD fluid inlet 196 into the open space defined by shell 194.

Outlet cover 200 likewise includes or defines a shell 202 providing a space for filtered fresh PD fluid or used PD fluid to be distributed across the shell before either (i) flowing as fresh PD fluid through a patient lumen 204 and transfer set-side connector 106 to the patient or (ii) flowing as used PD fluid through patient lumen 204 and used PD fluid port 104u of lumen-side connector 104 (FIG. 1) to used PD fluid lumen 54. Patient lumen 204 is formed with shell 202 in one embodiment. As illustrated in FIG. 12, shell 202 defines an opening 206 that allows filtered fresh PD fluid to flow from shell 202 into patient lumen 204 on its way to the patient via transfer set-side connector 106. It should be appreciated that while used PD fluid could theoretically flow from patient lumen 204, through opening 206 into shell 202, the negative pressure applied to used PD fluid port 104u to remove used PD fluid from the patient provides little incentive for the used PD fluid to do so.

Membrane modules 210a, 210b . . . 210n each include a frame 212a or 212b that defines an opening 214. Opening 214 in the illustrated embodiment is partially filled with support ribs 216 that support a filter membrane 112a, 112b. Ribs 216 do not completely block opening 214 and thus allow fresh PD fluid to be filtered across filter membranes 112a, 112b from the inlet cover 192. Filter membranes 112a, 112b are ultrasonically sealed, heat sealed and/or adhesively sealed, e.g., via solvent bonding, to an inner perimeter of frame 212a or 212b so as to cover opening 214 and be supported by ribs 216. After a desired number of membrane modules 210a, 210b . . . 210n are stacked together (could be only a single module or two or more modules), inlet and outlet covers 192, 200 are fitted to the exposed surfaces of the one or more membrane module 210a, 210b . . . 210n, after which the modules and covers are ultrasonically sealed, heat sealed and/or adhesively sealed, e.g., via solvent bonding, to form modular filter set 100 of the third primary embodiment.

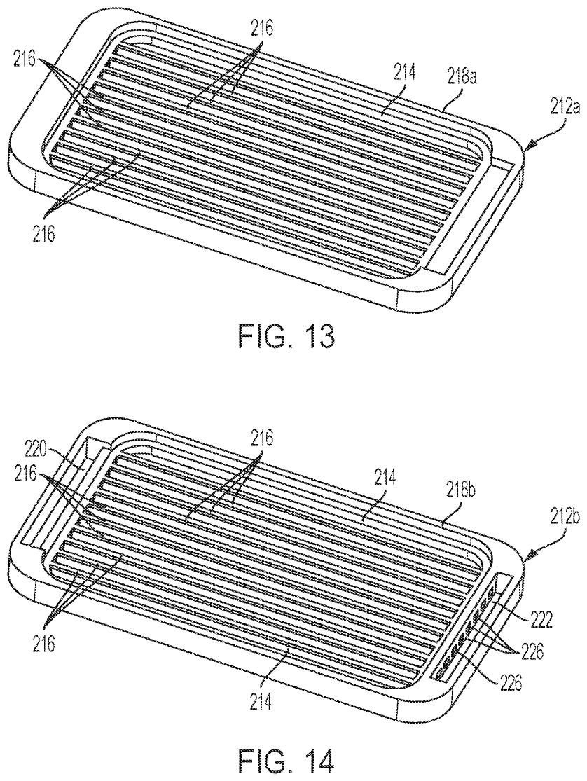

FIG. 13 illustrates frame 212a in more detail, while FIG. 14 illustrates frame 212b in more detail. Frame 212a and frame 212b each define opening 214, which is partially filled with support ribs 216 that support a filter membrane 112a, 112b. Opening 214 for frame 212a is defined by a perimeter 218a, from which support ribs 216 extend. Opening 214 for frame 212b is defined by a perimeter 218b, from which support ribs 216 extend.

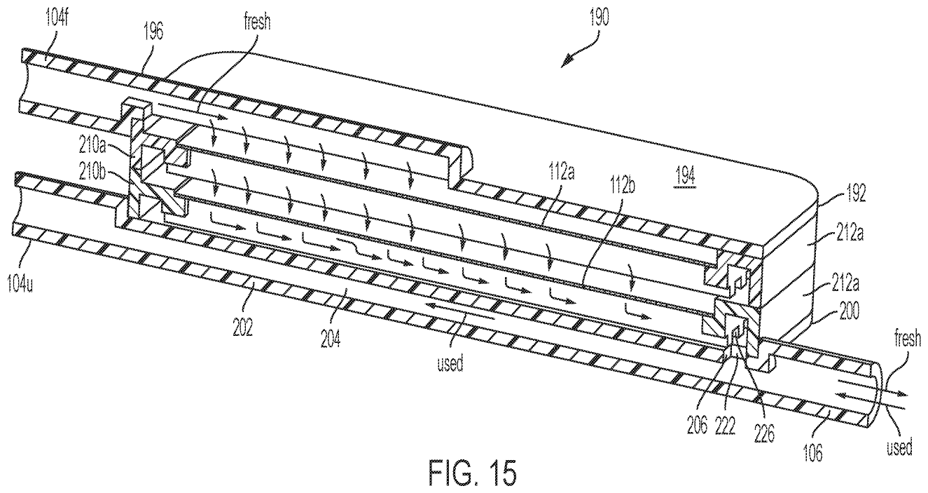

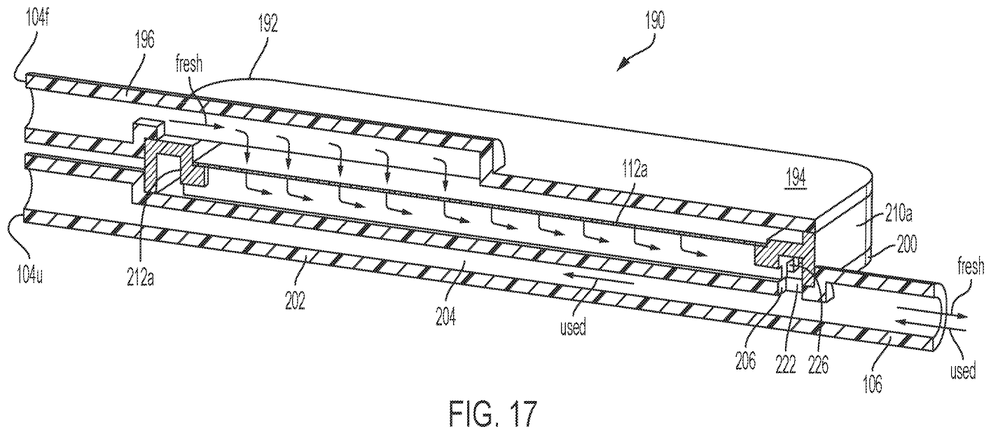

Perimeter 218a for frame 212a is solid such that all fresh PD fluid incoming from left to right is forced over and through filter membrane 112a, 112b and thereafter between support ribs 216. Frame 212a is open beneath ribs 216, such that fresh PD fluid filtered through filter membrane 112a, 112b extends to whatever structure resides beneath frame 212a, e.g., a second frame 212b (FIG. 15) or outlet cover 200 (FIG. 17).

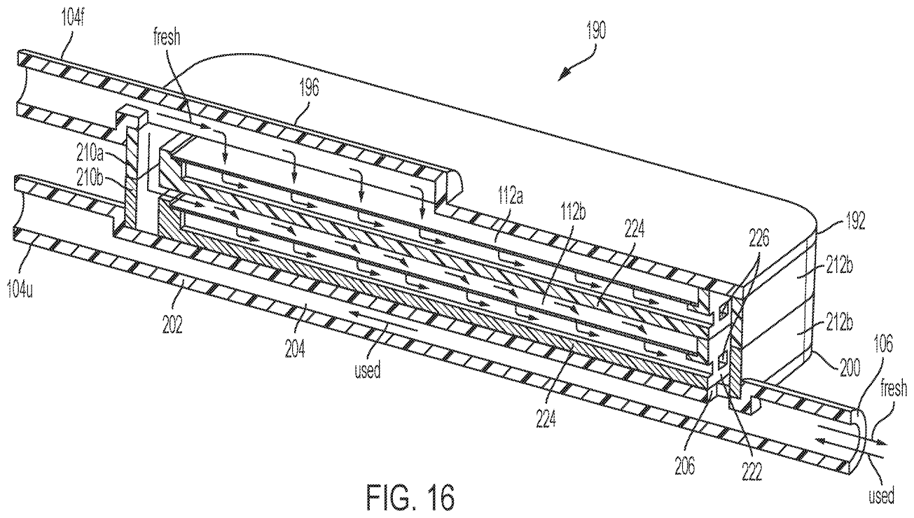

Perimeter 218b for frame 212b instead includes an inlet opening 220 and an outlet opening 222. Inlet opening 220 allows incoming fresh PD fluid to be split between (i) being forced over and through filter membrane 112a, 112b and thereafter between support ribs 216 and (ii) bypassing the filter membranes and flowing downward through inlet opening 220, e.g., to a second, lower frame 212b (FIG. 16). Frame 212b is not open beneath support ribs 216 and instead includes a bottom 224 (FIGS. 15 and 16) that traps filtered fresh PD fluid and forces the filtered fluid to flow across frame 212b and out through one or more aperture 226 into outlet opening 222. Filtered, fresh PD fluid then flows from outlet opening 222 either (i) to one or more other outlet opening 222 of a second frame 212b and then to outlet cover 200 (FIG. 16) or (ii) directly to outlet cover 200 (FIG. 15).

FIG. 15 illustrates that a redundant modular filter set 100 using body 190 is formed so that fresh PD fluid entering a first membrane module 210a of the stack (having frame 212a) via inlet cover 192 is filtered through filter membrane 112a of membrane module 210a (first filtration), flows to a second membrane module 210b of the stack (also having frame 212a), is filtered through filter membrane 112b of second membrane module 210b (redundant filtration), and flows out of second membrane module 210b via outlet opening 222 of second frame 212a and opening 206 of shell 202 into patient lumen 204 of outlet cover 200, and through transfer set-side connector 106 to the patient. Used PD fluid returns from the patient, through transfer set-side connector 106, patient lumen 204 and used PD fluid port 104u to used PD fluid lumen 54 (FIG. 1). The overall redundant modular filter set 100 using body 190 is similarly advantageous because it mitigates a compromised filter membrane and provides double filtration if both filter membranes are intact.

In an alternative embodiment for the redundant modular filter set 100 using body 190 of FIG. 15, second membrane module 210b of the stack instead includes a frame 212b, wherein the twice filtered fresh PD fluid flows instead along the top surface of the bottom 224 of frame 212b, through opening 206 of shell 202, into patient lumen 204.

FIG. 16 illustrates that a double membrane modular filter set 100 using body 190 is instead formed so that fresh PD fluid entering through the inlet cover 192 flows in parallel to the upper sides of both membrane modules 210a (having frame 212b), 210b (also having frame 212b), is filtered through both filter membranes 112a, 112b simultaneously or in parallel. It should be appreciated that the bottoms 224 of frames 212b, and the associated flow through apertures 226 into outlet opening 222, prevents PD fluid entering upper membrane module 210a from being filtered twice through both filter membranes 112a, 112b. Filtered PD fluid flows from the outlet openings 222 of frames 212b to patient lumen 204 of outlet cover 200, and then flows via patient lumen 204 and transfer set-side connector 106 to the patient. Used PD fluid again returns from the patient, through transfer set-side connector 106, patient lumen 204 and used PD fluid port 104u to used PD fluid lumen 54. The overall double membrane modular filter set 100 using body 190 also advantageously doubles the flow capacity of the filter set.

In an alternative embodiment, to allow membrane modules 210a, 210b to be manufactured the same and have only a single frame, it is contemplated to provide one or more pluggable hole or aperture (not illustrated) at the inlet end of the single frame to allow membrane modules 210a, 210b to operate in the redundant manner in FIG. 15 and the double filter membrane manner in FIG. 16. The one or more hole or aperture is plugged in each upper and lower module of the redundant version of FIG. 15, so that all incoming fresh PD fluid is forced over the top of upper membrane module 210a. The one or more hole or aperture is unplugged in upper module 210a and plugged in lower module 210b in the double filter membrane version of FIG. 16, so that incoming fresh PD fluid is split between upper and lower membrane modules 210a, 210b. The one or more hole or aperture would also be plugged in membrane module 210a of the single filter version of FIG. 17.

FIG. 17 illustrates that a single membrane modular filter set 100 using body 190 employs a single membrane module 210a in combination with the inlet and outlet covers 192, 200. Here, inlet and outlet covers 192, 200 are sealed to single membrane module 210a (having frame 212a) so that fresh PD fluid entering the membrane module via inlet cover 192 flows into an inlet compartment beneath shell 194 of inlet cover 192, is filtered evenly through filter membrane 112a of membrane module 210a into an outlet compartment formed by shell 202 of outlet cover 200, and flows out of the outlet compartment via outlet opening 222 of frame 212a and opening 206 of shell 202 into patient lumen 204 of outlet cover 200 to the patient. Used PD fluid again returns from the patient, through transfer set-side connector 106, patient lumen 204 and used PD fluid port 104u to used PD fluid lumen 54 (FIG. 1). The overall single membrane modular filter set 100 using body 190 likewise reduces size and cost.

Used PD fluid removed through the patient's transfer 58 in each of the above filter body embodiments under negative pressure from PD fluid pump 24 enters modular filter set 100 via transfer set-side connector 106. The used PD fluid then flows through the provided body 110, 150 or 190 via a corresponding patient lumen 134, 170, 204, and flows through used PD fluid port 104u and used PD fluid lumen 54, back to PD machine or cycler 20. PD machine or cycler 20 pumps the used PD fluid under positive pressure via PD fluid pump 24 to drain via drain line 60. The used PD fluid for the most part does not contact the underside of filter membranes 112a, 112b in bodies 110, 150 or 190. Filter membranes 112a, 112b accordingly remain viable over the course of multiple fills of a treatment prior to being discarded with modular filter set 100.

It should be understood that various changes and modifications to the presently preferred embodiments described herein will be apparent to those skilled in the art. It is therefore intended that any or all of such changes and modifications may be covered by the appended claims. For example, while a dual lumen patient line 50 is illustrated, a single lumen patient line may be provided alternatively.

1. A peritoneal dialysis (“PD”) system (10) comprising:
a PD machine (20);
a patient line (50) extending from the PD machine (20); and
a filter set (100) in fluid communication with the patient line (50), the filter set (100) including a filter body (110, 150, 190) housing first and second filter membranes (112a, 112b), the filter body (110, 150, 190) configured to be placed in different arrangements such that fresh PD fluid (i) flows through the first filter membrane (112a) and then through the second filter membrane (112b) or (ii) splits and flows through the first and second filter membranes (112a, 112b) in parallel.

2. The PD system (10) according to claim 1, wherein the filter body (110) includes a first membrane module (120a) housing the first filter membrane (112a), a second membrane module (120b) housing the second filter membrane (112b) and a fluidic spacer (130a, 130b) in fluid communication with the first and second membrane modules (120a, 120b), the fluidic spacer (130a, 130b) configured to cause (i) or (ii).

3. The PD system (10) according to claim 2, wherein the first membrane module (120a) is formed the same as the second membrane module (120b).

4. The PD system (10) according to claim 2, wherein for (i) the fluidic spacer (130a) is configured such that fresh PD fluid flows from the fluidic spacer (130a) through the first filter membrane (112a) of the first membrane module (120a), flows into the second membrane module (120b), flows through the second filter membrane (112b), and flows back into the fluidic spacer (130a) before flowing to a patient.

5. The PD system (10) according to claim 2, wherein for (ii) the fluidic spacer (130b) is configured such that fresh PD fluid splits from the fluidic spacer (130a) into the first and second membrane modules (120a, 120b), is filtered through the first and second filter membranes (112a, 112b) in parallel, and flows back into the fluidic spacer (130b) before flowing to a patient.

6. The PD system (10) according to claim 2, wherein the fluidic spacer (130a, 130b) includes a patient lumen (134) that allows filtered fresh PD fluid to flow to a patient and used PD fluid to flow from the patient.

7. The PD system (10) according to claim 2, wherein the fluidic spacer (130a, 130b) includes a blind fresh PD fluid inlet (132) that accepts fresh PD fluid from the patient line (50).

8. The PD system (10) according to claim 1, wherein the filter body (150) includes a first membrane module (160a) housing the first filter membrane (112a), a second membrane module (160b) housing the second filter membrane (112b), the first and second membrane modules (160a, 160b) including a plurality of inlets, passageways or lumens (166, 168, 170, 172) positioned and arranged to cause (i) and (ii), and wherein a portion of the inlets, passageways or lumens (166, 168, 170, 172) are plugged to cause (i) or (ii).

9. The PD system (10) according to claim 8, wherein the first membrane module (160a) is formed the same as the second membrane module (160b).

10. The PD system (10) according to claim 8, wherein at least one of the first or second membrane modules (160a, 160b) includes an upper compartment (162) and a lower compartment (164), the upper compartment (162) formed the same as the lower compartment (164).

11. The PD system (10) according to claim 8, wherein for (i) the plurality of inlets, passageways or lumens (166, 168, 170, 172) are plugged such that fresh PD fluid flows through the first filter membrane (112a) of the first membrane module (160a), flows into the second membrane module (160b), and flows through the second filter membrane (112b), before flowing to a patient.

12. The PD system (10) according to claim 8, wherein for (ii) the plurality of inlets, passageways or lumens (166, 168, 170, 172) are plugged such that fresh PD fluid splits into the first and second membrane modules (160a, 160b) and is filtered through the first and second filter membranes (112a, 112b) in parallel before flowing to a patient.

13. The PD system (10) according to claim 8, wherein the first and second membrane modules (160a, 160b) each include a patient lumen (170), and which for (ii) includes a bridge connector (180) configured to divert fresh PD fluid from one of the patient lumens (170) to the other of the patient lumens (170).

14. The PD system (10) according to claim 1, wherein the filter body (190) includes a first membrane module (210a) housing the first filter membrane (112a), a second membrane module (210b) housing the second filter membrane (112b), the first and second membrane modules (210a, 210b) stacked together, the filter body (190) further including an inlet cover (192) and an outlet cover (200), at least one of the first and second membrane modules (210a, 210b), the inlet cover (192) and the outlet cover (200) configurable to cause (i) or (ii).

15. The PD system (10) according to claim 14, wherein the first membrane module (210a) and the second membrane module (210b) employ at least one of a first frame (212a) that forces all incoming fresh PD fluid to be filtered through an associated first or second filter membrane (112a, 112b) or a second frame (212b) that filters a portion of the incoming fresh PD fluid through the associated first or second filter membrane (112a, 112b) and allows another portion of the incoming fresh PD fluid to bypass the associated first or second filter membrane (112a, 112b).

16. The PD system (10) according to claim 1, wherein the patient line (50) is a dual lumen patient line including a fresh PD fluid lumen (52) placed in fluid communication with a fresh PD fluid port (104f) of the filter set (100), the dual lumen patient line (50) further including a used PD fluid lumen (54) placed in fluid communication with a used PD fluid port (104u) of the filter set (100).

17. The PD system (10) according to claim 1, wherein the filter set (100) is configured to connect directly to a patient's transfer set, or wherein the filter set (100) includes a flexible tube (108) configured to connect to the patient's transfer set.

18. The PD system (10) according to claim 1, wherein the PD machine (20) includes a pressure sensor (28b) positioned and arranged to sense a pressure of fresh PD fluid downstream from the filter membrane (112) during a patient fill.

19. The PD system (10) according to claim 1, wherein the first and second filter membranes (112a, 112b) are sterilizing grade or bacteria reduction filter membranes.

20. A peritoneal dialysis (“PD”) system (10) comprising:
a PD machine (20);
a patient line (50) extending from the PD machine (20); and
a filter set (100) in fluid communication with the patient line (50), the filter set (100) including a filter body (110, 150, 190) and at least one of a first membrane (112a) or a second filter membrane (112b), the filter body (110, 150, 190) configured to be placed in different arrangements such that fresh PD fluid (i) flows through the first filter membrane (112a) and then through the second filter membrane (112b), (ii) splits and flows through first and second filter membranes (112a, 112b) in parallel, or (iii) flows through the first filter membrane (112a) only.
